(19) United States
(12) Patent Application Publication
(10) Pub. No.: US 2007/0173830 A1
(43) Pub. Date: Jul. 26, 2007

(54) Method of percutaneous paracoccygeal pre-sacral stabilization of a failed artificial disc replacement

(76) Inventor: Charles D. Rosen, Manhattan Beach, CA (US)

Correspondence Address: FULWIDER PATTON LEE & UTECHT, LLP, 200 OCEANGATE, SUITE 1550, LONG BEACH, CA, 90802, US

(21) Appl. No.: 11/499,999

(22) Filed: Aug. 7, 2006

Related U.S. Application Data
(63) Continuation-in-part of application No. 11/335,267, filed on Jan. 19, 2006.

(51) Int. Cl. A61F 2/30

(52) U.S. Cl. 606/61

(57) ABSTRACT

A procedure for stabilization in situ of a failed artificial disc replacement (ADR) using a pre-sacral paracoccygeal approach to an inter-vertebral disc space, such as the L5-S1 disc space for example, where a bore is created in the ADR using two counter rotating small drills, and then a larger hollow drill over this to create a tunnel. Through this bore a hollow tube with a compressive fastener is inserted and used to compress the endplates of the ADR. The fastener may have ends that prevent movement of the fastener once established in the ADR, and maintain the ADR in compression. Then the tube is filled with material to grow bone and fuse one vertebrae to the other through the tube. Subsequent to the anterior stabilization and fusion of the ADR, a posterior spinal fusion operation can be performed with the stabilized ADR such that regenerative growth of bone can surround and form over the ADR without relative movement of the ADR to resist complete fusion and immobilization, and thus to improve the clinical results.

### CROSS-REFERENCES TO RELATED APPLICATIONS

This application is a continuation in part of U.S. patent application Ser. No. 11/335,267 filed Jan. 19, 2006, to which priority is claimed, and to the contents of which are fully incorporated by reference herein.

### BACKGROUND OF THE INVENTION

The present invention relates generally to spinal column reconstruction procedures, and more particularly to a procedure for stabilizing an artificial disc replacement (ADR) in situ using a percutaneous paracoccygeal pre-sacral approach. This is performed for the specific purpose of improving the clinical results of a concurrently performed posterior fusion in the situation where the ADR has failed.

Lumbar disc replacement surgery has recently become an available surgical alternative to lumbar spine fusion, although the development of the procedures and the prostheses themselves are in their infancy, particularly for use in the United States. Presently, disc replacement surgery is proposed only for single-level, painful degenerative disc disease that has failed to improve after at least six months of intense spine-focused rehabilitation in a patient without significant physical or psychological contraindications. Candidates are presently diagnosed with degenerative disc disease (DDD) or post-laminectomy syndrome at either the L4-L5 or L5-S1 levels of the lumbar spine, but not both, although other levels of the spine are also theoretically possible.

Artificial discs, such as the Charite™ artificial disc manufactured by DePuy Spine, Inc., 325 Paramount Drive Raynham, Mass. 02767, were approved by the FDA in October, 2004. The object of the artificial disc is to restore the intervertebral disc height and neuroforaminal height while restoring physiologic motion. The disc insertion is performed anteriorly through a small incision in the abdomen. The patient's organs are displaced to the side so that the surgeon can visualize the spine while shielding important anatomic structures. The collapsed or degenerated disc is removed and the prosthetic artificial disc is inserted in the spinal column in its place. The prosthesis is formed of two metal plates made of a cobalt chrome alloy or other suitable biocompatible material sandwiching a plastic (ultra-high molecular weight polyethylene or UHMWPE) core. During the replacement procedure, the two endplates are pressed into the vertebrae above and below the disc space. The end plates are formed with teeth on the outer surface that help secure the prosthesis to the adjoining bone. The plastic core and endplates serves to restore the proper distance between the two vertebrae (disc height), and simulate the resiliency of the natural disc. The theory behind the disc replacement surgery is that the artificial disc stays in place by the spinal ligaments and remaining part of the annulus of the disc, as well as the compressive force of the spine.

Unfortunately, the success rate of the ADR surgery has been less than optimal, with a large percentage of ADR patients experiencing severe and chronic pain after the surgery. The present inventor voiced doubts at the time the FDA approved the ADR about the safety and reliability of the new disc replacement surgery, doubts that have become realized by the large number of patients who have experienced tremendous pain and complications with their new disc replacements. One major complication experienced by a large majority of patients is that the disc fails to bond properly in the spinal column, resulting in instability or dislocation/subluxation of the disc and the accompanying disabilitating pain. The ADR may increase the motion of the facet joints, leading to subsequent degeneration and pain. Fractures of various parts of the vertebra may also occur during or after the implantation, as well as fractures of the polyethylene core. Some cases of chronic debilitating pain may not have any obvious cause but still constitute a failure of the ADR. The widespread failure of these discs has become so prevalent that it became apparent to the present inventor that a better salvage procedure was needed where the disc is stabilized in some fashion prior to an attempt at posterior fusion. Removal of the ADR is a poor and dangerous alternative due to the life threatening consequence of exsanguination and death from tearing of scarred down large vessels. Thus, stabilization by the method of the present invention was developed to increase the clinical success rate of a salvaging fusion procedure done posteriorly.

As a result of examining the various complications of the ADR, a new mathematical model of spinal motion was developed that appears to be a more accurate depiction of spinal motion than the model used in developing the ADRs presently on the market which have the center of rotation assumed to be in the front of the spinal canal. The new model suggests that this assumption is erroneous, and that the center of rotation of the lumbar vertebral segments is posterior to the spinal canal.

The purpose of the stabilization procedure is to allow for a posterior fusion, as well as provide for an anterior fusion through the ADR without the necessity of removing the ADR. A posterior fusion is attempted by using bone graft or bone substitutes to promote the vertebra to fuse together. Presently, when a fusion has been attempted for a failed ADR the results have been poor with a sixty percent (60%) failure rate (defined as continuing pain). Sometimes fusion occurs and pain is still present, and many other times fusion is unsuccessful. Without the ADR, posterior fusion has a success rate of over eighty percent (80%), so the presence of the ADR has a dramatic effect on the success rate of the fusion surgery. The present inventor has proposed a safe procedure to dramatically increase the success rate of the posterior fusion when an ADR is present.

### SUMMARY OF THE INVENTION

The present invention proposes that a stabilization of the ADR prior to attempting a posterior fusion will promote the fusion process by encouraging regenerating bone material to grow around and through the ADR to fortify the spine structure. Stabilization of a floating or loose ADR is performed percutaneously by a pair of simultaneously rotating small diameter drills. This involves providing for an anterior stabilization, as well as adding an anterior fusion, via a subsequently drilled hole in the ADR. Material to stimulate bone growth, such as bone graft or allograft like Bone Morphogenetic Protein (BMP), is placed in the hole that fuses the ADR to the spinal column and thereby supplements the stabilization of the device. The approach to the lumbar spine is paracoccygeal in the area posterior to the mesorectum and anterior to the sacrum to avoid the scarred area of the iliac vessels. The L5-S1 disc space, for example, can be accessed by drilling through a cannula that protect the rectum and intestines. This same method described here can be used for the L4-L5 or L3-L4 space as well.

The drill is used to pierce the metallic base plates of the ADR to create a through and through bore, with irrigation maintaining a proper environment at the drilling surface. In a preferred method, two small diameter drills, spinning in the same direction to neutralize overall torque, form two small holes. Suction and evacuation of the debris generated by the drilling operation may be conducted simultaneously with the drilling by water being pumped in through an inflow portal and being vacuumed out through an outflow portal. This process also provides for cooling of the drill bits. After drilling through the ADR, both drill bits are left in place to provide stabilization for subsequent steps. The next step involves a hollow annular drill bit that drills out a tunnel over the two drill bits left in place, similar to a dowel. The edges of the drill may preferably be of a diamond or carbon material. The drill is an oscillating drill irrigation for heat control and debris removal continue to be done through the inflow and outflow portals. Removal of the cylindrical segment or “dowel” of ADR provides for a tunnel connecting the bone of the vertebrae below the ADR with that the vertebrae above. Then a fastener capable of adjustable compression is placed into the bore ADR to compress the disc in situ and stabilize the disc in the spinal column. This fastener may be of the form of a pop rivet that is deployed to firmly anchor into the vertebrae at each end of the tunnel, thus stabilizing the ADR. Subsequently, bone generating material is placed in the tunnel to create a fusion of bone going from one vertebrae to the other to provide further long term stability.

Subsequently, a posterior spinal fusion, and decompression, if either one or both is needed, is performed with rigid fixation. This allows regenerative bone to grow in from the posterior aspect, in addition to the anterior fusion, and thus permanently address the instability or other causes noted above that may be the root of pain from the failed ADR.

### BRIEF DESCRIPTION OF THE DRAWINGS

Other features and advantages of the invention will become apparent from the following detailed description, taken in conjunction with the accompanying drawings which illustrate, by way of example, the features of the invention:

FIG. 1 is a lateral view, partially in shadow, of a patient prone on the table with fluoroscopes in place and guidewire needle inserted;

FIG. 2 is a view of the insertion of the blunt trocar into the S1 disc space;

FIG. 3 is a top view of the insertion of the blunt trocar into the S1 disc space;

FIG. 4 is a side view of the insertion of the drill into the ADR;

FIG. 5 is a top view of the insertion of the twin smaller drills into the ADR;

FIG. 6 is a perspective view of the L5-S1 disc space with the ADR in place showing the two drills through the ADR;

FIG. 7 is a perspective view of the L5-S1 disc space with the ADR being about to be drilled by a large bore annular drill;

FIG. 8 is a perspective view of the L5-S1 disc space after the drilling by the large diameter drill to create a dowel segment of the ADR;

FIG. 9 is a perspective view of the L5-S1 disc space with a large diameter bore retracted with withdraw a dowel segment from the center of the ADR;

FIG. 10 is a perspective view of the L5-S1 disc space with a tube and insertion of a fastener through the bore created by the large diameter drill;

FIGS. 11-14 are perspective views of the L5-S1 disc space with a various embodiments of alternate fasteners inserted into the bore and the proximal end tightened to place the ADR in compression;

FIG. 12 is an enlarged perspective view of the first embodiment of the fastener in the undeployed and deployed positions;

FIG. 15 is a perspective view of a mechanism for drilling two introductory holes into the ADR using first and second counter rotating drill bits;

FIG. 16 is a top view of the drill guide for use with the mechanism shown in FIG. 15;

FIG. 17 is a perspective view of the drill guide and drill mechanism of FIGS. 15 and 16;

FIG. 18 is a perspective view of the drill guide and drill mechanism of FIG. 17 with a hollow annular drill bit for cutting a dowel into the ADR;

FIG. 19 is an enlarged perspective view of the hollow annular drill bit of FIG. 18; and

FIG. 20 is a cross-sectional schematic view of the pop rivet and ADR for compressing the ADR.

### DETAILED DESCRIPTION OF THE PREFERRED METHODOLOGIES

Described below is a method for in situ stabilization of a failed ADR prior to a posterior fusion procedure. The stabilization employs a novel paracoccygeal percutaneous approach that is far safer than an anterior approach and permits greater fusion opportunity due to immobilization of the failed ADR. Prior to stabilization, it may be preferable to employ a postero-lateral approach described herein where it has been determined that there is a need to retrieve a dislocated or subluxed ADR prior to stabilization, or because direct visualization is desired through the endoscope of the concurrent stabilization procedure through the pre-sacral approach. Access through one or more poster-lateral portals may also assist also in evacuating debris that results from the drilling procedure and in cooling the drill with irrigation.

Percutaneous posterolateral endoscopic access to a failed ADR disc space requires initially the establishment of key fluoroscopic landmarks using the fluoroscopes in the AP and lateral plane. These landmarks are the center of the disc, the area of the disc centered just lateral to the pedicle, and the disc angle line that bisects the disc in the lateral projection. The skin entry is determined from the inclination of the failed disc. The lateral location of the skin incision's from the midline determines the trajectory angle into the particular disc space of the ADR just lateral to the pedicle, the basal part of each side of the neural arch of a vertebra connecting the laminae with the body.

A long guide wire 20 is laid across the patient in the anteroposterior (AP) plane and the fluoroscope is used to locate the midline of the disc in the AP plane. A pen mark is placed on the skin is used to demarcate the position. Then the guidewire 20 is placed transversely over the disc and this is position is also marked. The intersection of the lines is the center of the disc. It is important to obtain a true AP line and a true lateral line of the selected disc space being visualized so that both endplates of the ADR are precisely parallel. The entry point for a spinal needle is then estimated to be on the axis of the transverse line with a trajectory of about 30 to 40 degrees off the midline. At L5/S1 juncture, the angle of attack may be steeper to avoid the iliac crest. This estimation is roughly 4 fingerbreadths lateral of the midline. However, the main way of guiding the needle to enter the disc just lateral to the facet joint is by tracking the progress of the needle using the fluoroscopes and re-adjusting the trajectory as needed in both planes until the desired location is hit. In some instances, hitting the facet and “walking off” the needle laterally can be helpful and confirmatory of location. Monitoring with intra-operative continuous evoked potentials and EMG's help to prevent inadvertently injuring the nerve root, which can be employed with EMG feedback prior to entering the disc (such as done with pedicle screw testing).

The hollow needle with a stylet is advanced into the disc space of the ADR. Once the needle is in place and the location confirmed, a guide wire 20 is then exchanged for the stylet. A blunt dilator is then advanced over the guidewire, and a working cannula is advanced over the guidewire. The blunt dilator and guide wire are removed once the working cannula is sufficiently deep into the annulus. The endoscope is then inserted into the working cannula for visualization. Subsequent work to reduce the disc, if needed, can be done either through the same cannula as the endoscope, or through a separate and identical portal to the disc but on the other side. A cannula portal on the other side established in the same fashion can be used to remove debris from the drilling operation or to provide irrigation for same if necessary.

To reduce a dislocated or subluxed ADR, a number of methods may be tried with the simplest first. A sharp claw may be set into the polyethylene midsection of the disc or latched on the metal plate in effort to pull the ADR back into place. If this fails, then an acrylic glue may be applied to the ADR since such a glue can be adherent to polyethylene. A small amount of glue is pushed through a spinal needle, or the like, that is in contact with the ADR and allowed to set, then reduction attempted by manipulating the spinal needle with the adhered ADR. Failing this, the ADR may be penetrated by drilling and screwing a threaded member into the ADR. For example, a threaded sharp trocar point guide wire may be used to attempt to insert directly into the polyethylene. If penetration is difficult then drilling first may be needed. If the metal endplate is also dislocated or subluxed, then initial drilling will almost certainly be required.

Whether reduction of a dislocated ADR is required initially or not, the postero-lateral portal(s) can be used to assist in the stabilization of the ADR that will commence through the pre-sacral approach and portal concurrently. The accessory postero-lateral portal(s) is useful for visualization of the progress of the stabilization. Suction for debris removal as well as irrigation can be accomplished using one portal for each. If only one accessory portal is used, then intermittent or continuous suction and irrigation can be done simultaneously through therein.

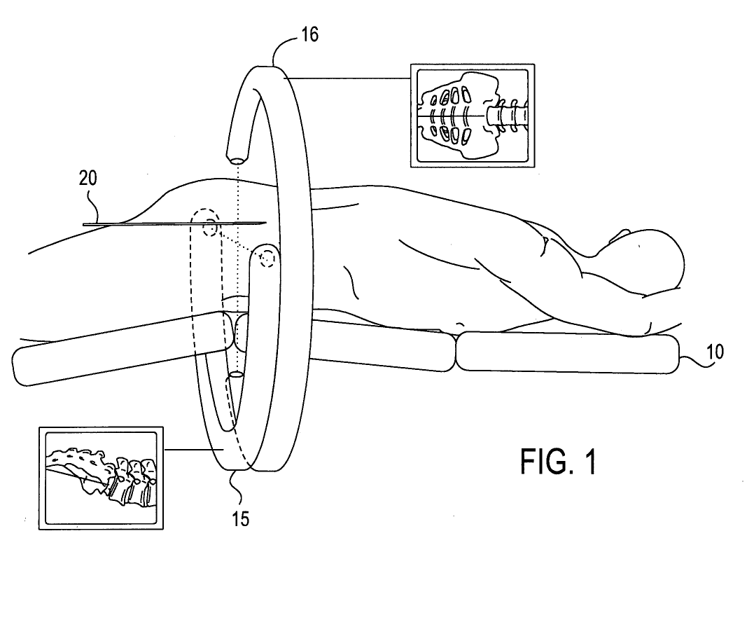

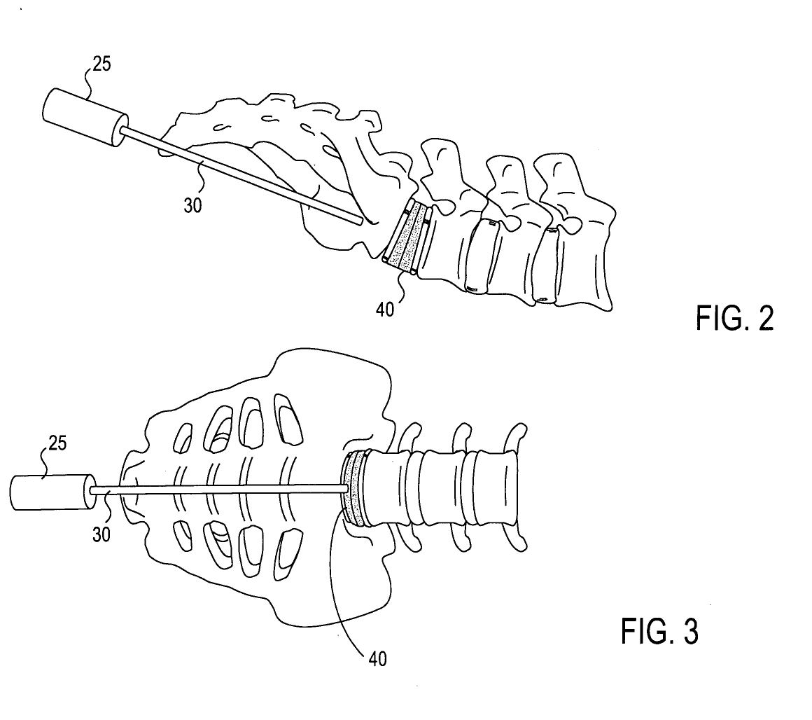

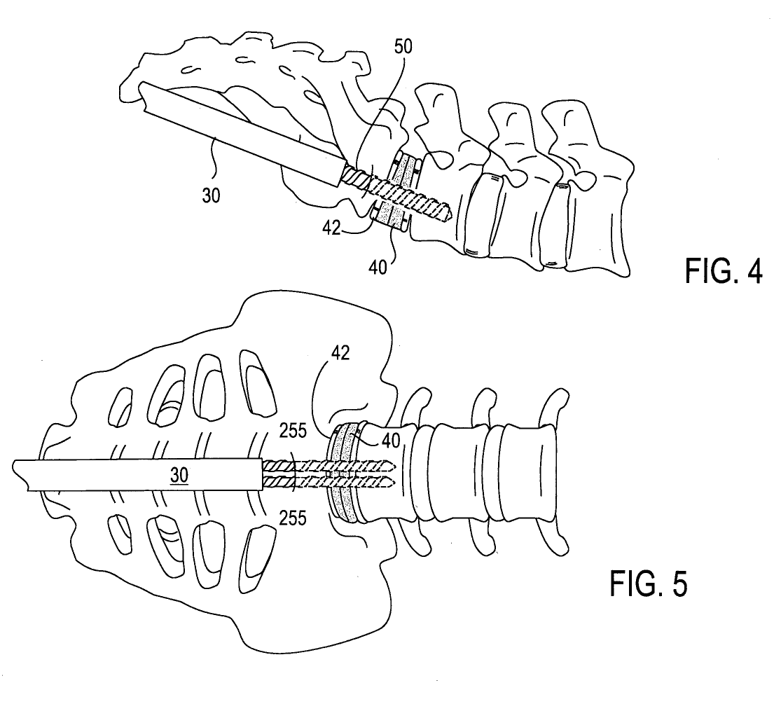

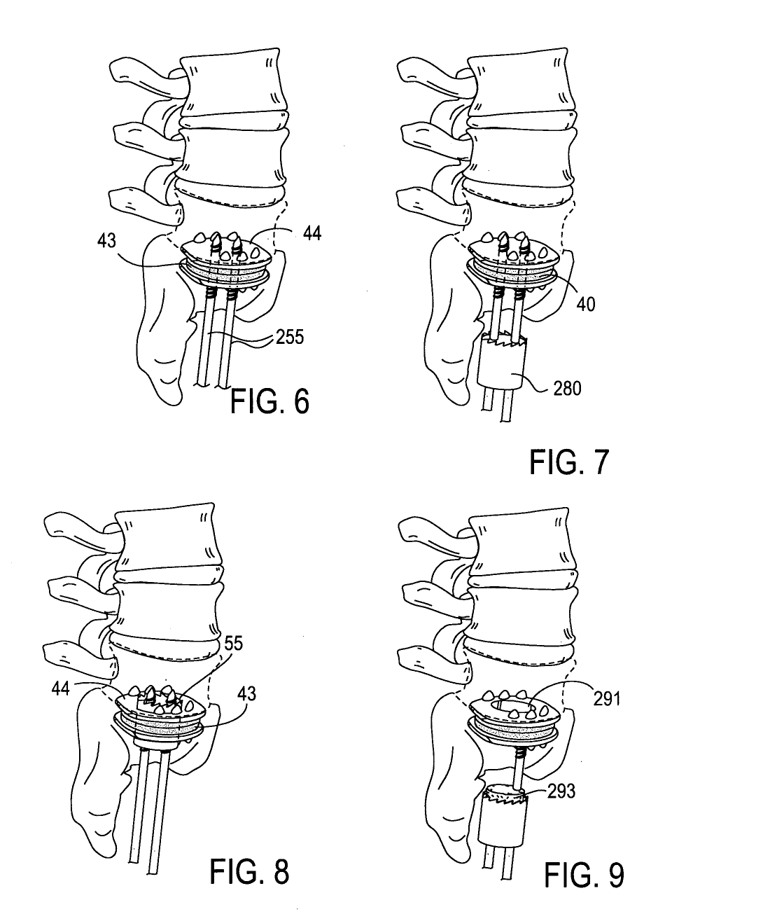

The procedure for stabilizing the ADR in situ will now be described. First, the pre-sacral approach to the ADR stabilization is initiated by prepping the area around the patient's anus with a betadine wash and then antiseptic paint. The area is draped off, and a standard surgical prep of the sacrococcygeal area and lumbar spine area are perforned. As shown in FIG. 1, the patient is prone on a Jackson table or similar table 10 with a slight flexion of hips to improve the exposure of the sacrococcygeal area. First and second C-arm fluoroscopes 15,16 are positioned such that the first fluoroscope 16 is aligned in AP plane and the second fluoroscope 15 is aligned in lateral plane. Once the scopes are in place and their orientations confinred, a 1.5 to 2.0 cm incision is made through the skin and subcutaneous fascia 1-2 cm caudal to the left or right of the tip of the coccyx and 2 cm superior to it. A cannula and blunt trocar is passed through the incision and located using the fluoroscopes to the L5-S1 disc area. As shown in FIGS. 2 and 3, a blunt trocar 25 and cannula 30 is inserted through the incision until the distal end of the cannula 30 is positioned on the anterior midline of sacrum. At this point, the fluoroscopes 15,16 in the AP and lateral planes are checked and the position of the blunt trocar 25 and cannula 30 are confirmed.

Once the fluoroscopes are checked, the blunt trocar 25 and working cannula 30 are advanced along the anterior sacrum with care to maintaining constant contact with the skeletal structure up to a position just below L5/S1 disc space. The trajectory of the blunt trocar and the cannula are once again confirmed using the AP and Lateral fluoroscopy. At this point, the blunt trocar 25 may be retracted and replaced with sharp guide pin (not shown) that is used to tap into the sacrum until it reaches the proximal base plate 42 of failed ADR 40.

It is preferable at this point to dilate the soft tissue and boney entry at the sacrum with dilators in 2 mm increments, beginning with 6 mm and concluding with a 10-12 mm working cannula 30 that is docked into the sacrum. With the entrance to the sacrum dilated, a drill is inserted into the cannula 30 until it bears against the endplate 42 of the ADR 40. Checking and confirming the orientation of the drill so as to be orthogonal, or within 45 degrees of this, to the plane of the ADR end plate 42 and centered in the face of the endplate, or off center as long as projected trajectory includes both metal plates of the ADR, the drill penetrates the ADR 40.

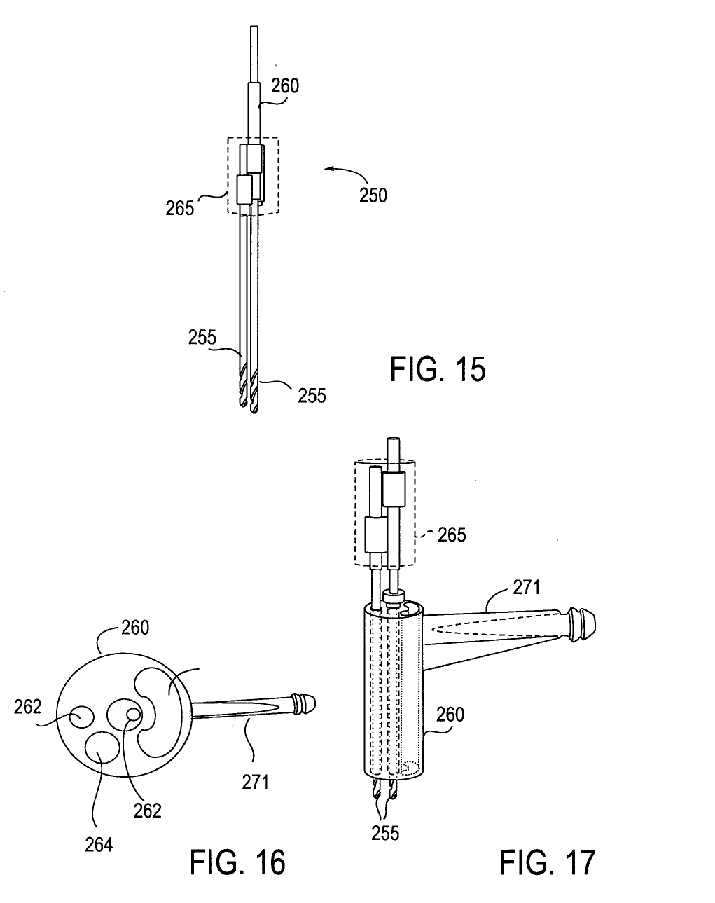

One of the problems with drilling hard surfaces such as the metal end plates of the ADR is the tendency for the ADR to twist or rotate during the drilling. In the present situation, the location of the ADR within the spinal column makes it effectively impossible to reliably secure the ADR and prevent this twisting. Because the rotation of the ADR could cause extreme damage to the surrounding spinal structure and other tissues, blood vessels, etc., a safe method of drilling the necessary hole is needed. One proposed solution is the use of paired small diameter drills used simultaneously in a parallel relationship. FIGS. 15-17 illustrates a mechanism 250 suitable for the present purpose having first and second drill bits 255 adapted to turn in opposite directions. The drill bits 255 extend through a drill guide 260 from a gear box 265. The drill guide 260 is preferably equipped with a drill portals262, an irrigation portal 264, and a suction portal 270 leading to a suction channel 271 that doubles as a rotation control handle.

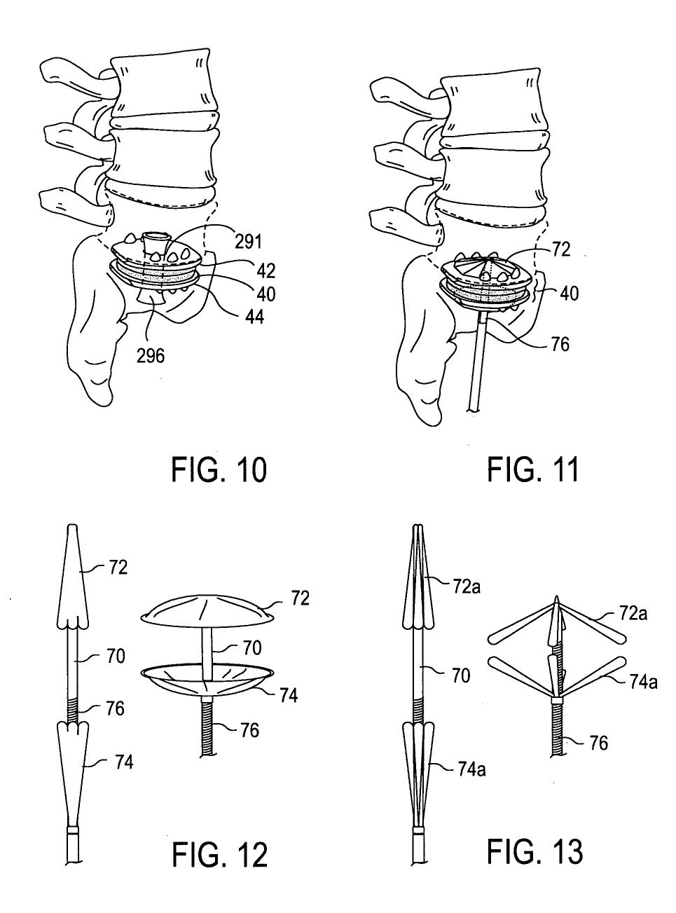

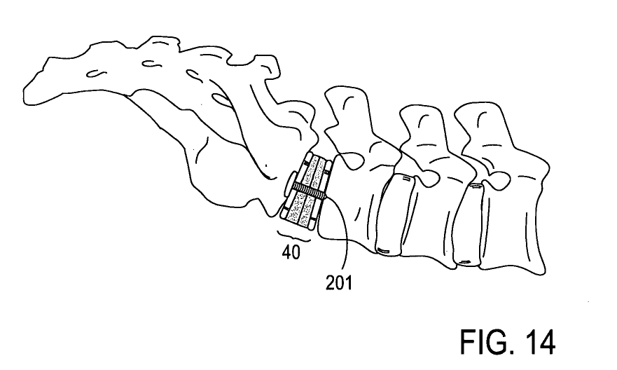

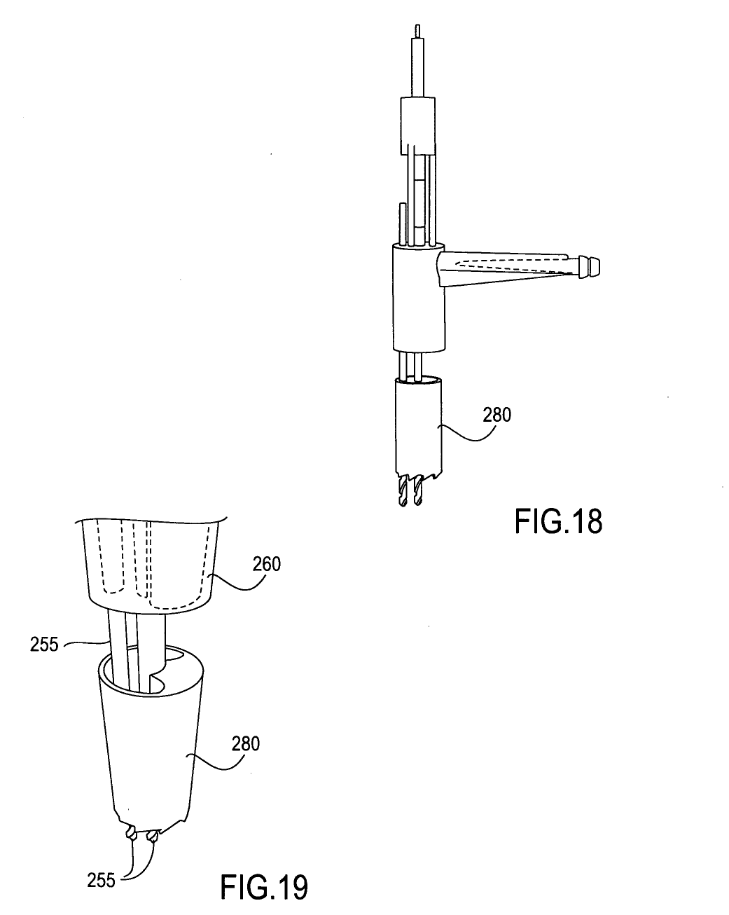

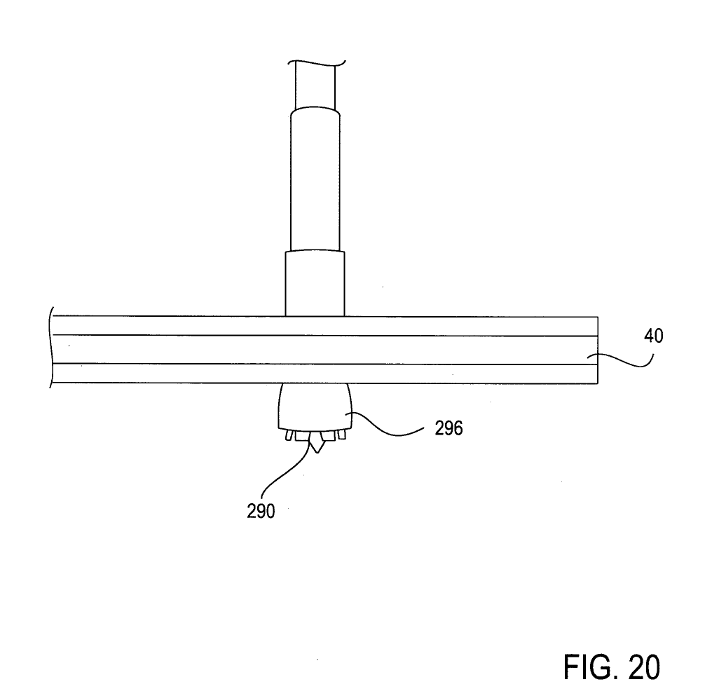

After the double bore of both drills 255 in the ADR 40 have been established (see FIGS. 5,6), it may be desirable to move the prosthesis prior to stabilization without making a new incision. A reduction tool may be used is move the disc. This reduction tool, as pictured, grasps one of the small diameter drills that has been left in place in order to maneuver the dislocated ADR into its proper position prior to stabilization. Re-drilling different holes and re-maneuvering can be repeated if the ADR is not satisfactorily positioned the first time. With the ADR properly positioned, a larger circular drill 280 having an annular hollow blade with diamond or carbon cutting edges (FIGS. 18,19), is placed over the two smaller drills 255 left in place to prevent torque. Using the large diameter annular drill 280, a cylindrical “dowel” of ADR can be drilled and removed from both end plates (FIGS. 7-9), creating a continuous tunnel 291 from one vertebrae to the other on opposite sides of the prosthesis 40. A stabilizing connecting tube 296 is inserted in the tunnel (FIG. 10) and anchored at both ends using a pop rivet 290 such as that shown in FIG. 20. The pop-up rivet 290 passes through the tunnel 291 of the ADR and expands at respective ends to compress the disk 40 together and arrest relative movement of the end plates 42, 44. Other fasteners such as those shown in FIGS. 11-14 or others known in the art can be used to stabilize the disc.

With the pop rivet 290 or other fastener in place, the relative movement of the ADR's endplates 42, 44 are restricted. The tunnel in the large bore 291 is then filled with material to stimulate bone growth resulting in a solid channel of bone from one vertebrae to the next and going through the ADR. Such material could be that of Bone Morphogenetic Protein (BMP) or the like, or could be the patient's own bone grafted from another area. The ADR is stabilized such that the subsequent spine fusion procedure done in a standard fashion from behind or posteriorly results in additional stability. This posterior fusion would use standard pedicle screws and rods for stabilization and along with bone graft or BMP. If a decompression to relieve pressure on nerves is required then this would be done before. The ultimate goal is to have the rehabilitation/healing not be frustrated by movement of the ADR. The inventor stresses that other fasteners or stabilizing techniques may be consistent with the present invention, and the invention should not be limited to only those described herein. Rather, it is envisioned that one of ordinary skill in the art given the Applicant's disclosure herein could devise equivalent fasteners that would work equally as well as those described herein and such alternative embodiments should be considered part of the present invention.

I claim:

1. A method for in situ stabilization of an artificial replacement disc located in an inter-vertebral disc space comprising the steps of:
using a blunt trocar and cannula to establish a pathway along an anterior sacrum;
advancing the blunt trocar and cannula to a position just below the disc space on the sacrum;
dilating an entry of the sacrum with a working cannula docked to the sacrum;
passing first and second rotating drills simultaneously through the working cannula and dilated sacrum until said drills bear against an end plate of the ADR;
creating first and second bores through the ADR using the first and second drills;
drilling a larger hole around said first and second bores using a larger hollow drill to remove a cylindrical section of ADR including the first and second bores; and
inserting a hollow tube and fastener into said larger hole, and compressing the ADR with the fastener.

2. The method of claim 1 wherein bone stimulating material is placed in the tube to allow for a tunnel of fusion bone to grow and permanently connect vertebra adjacent said ADR

3. The method of claim 1 wherein the fastener is a pop rivet mechanism.

4. The method of claim 1 wherein said steps are followed by a spine fusion procedure for additional stability.

5. The method of claim 2 wherein said bone stimulating material is Bone Morphogenetic Protein (BMP).

6. The method of claim 2 wherein said bone stimulating material is bone material taken from a patient's body.
